(19) United States
(12) Patent Application Publication
(10) Pub. No.: US 2025/0253064 A1
(43) Pub. Date: Aug. 7, 2025

(54) INFORMATION PROVIDING APPARATUS, INFORMATION PROVIDING METHOD, AND RECORDING MEDIUM

(71) Applicant: NEC Corporation, Tokyo (JP)

(72) Inventor: Hiroaki YAMAMOTO, Tokyo (JP)

(73) Assignee: NEC Corporation, Tokyo (JP)

(21) Appl. No.: 18/942,986

(22) Filed: Nov. 11, 2024

(30) Foreign Application Priority Data
Feb. 6, 2024 (JP) 2024-016535

(51) Int. Cl. G16H 80/00; G16H 10/60; G16H 20/10

(52) U.S. Cl. CPC G16H 80/00; G16H 10/60; G16H 20/10

(57) ABSTRACT

An information providing apparatus includes a memory storing instructions and one or more processors configured to execute the instructions to: identify a customer; acquire information regarding a prescription issued to the identified customer and information indicating a work status of medicine dispensing based on the prescription; predict a time for the customer to wait based on at least one of the information regarding the prescription and the information indicating the work status of medicine dispensing based on the prescription; and output information to be provided to the customer in a case where the predicted time for the customer to wait exceeds a threshold value.

This application is based upon and claims the benefit of priority from Japanese Patent Application No. 2024-016535, filed on Feb. 6, 2024, the disclosure of which is incorporated herein in its entirety by reference.

### TECHNICAL FIELD

The present disclosure relates to an information providing apparatus, an information providing method, and a program, and, particularly, to an information providing apparatus, an information providing method, and a program for providing, to a customer, information for using a time to wait by the customer.

### BACKGROUND ART

Japanese Patent Application Laid-open Publication No. 2002-183306 discloses a technique related to the present disclosure. In the related technique, a plurality of terminal devices used in dispensing pharmacies and a plurality of terminal devices used in hospitals are coupled to each other via a communication network. Prescription data is transmitted from one of the terminal devices, which is used in one of the hospitals, where a patient has undergone examination, to one of the terminal devices, which is used in one of the dispensing pharmacies, where the patient receives a prescription medicine. In the one of the dispensing pharmacies, a pharmacist refers to the prescription data received by the one of the terminal devices, which is used in the one of the dispensing pharmacies, to dispense a prescription medicine. When the patient visits the one of the dispensing pharmacies, the pharmacist delivers the prescription medicine to the patient in exchange for a prescription in a form of a piece of paper, which has been issued by the one of the hospitals.

### SUMMARY

In view of such a situation described above, an object of the present disclosure is to provide, to a customer waiting for delivery of a prescription medicine, information for using a time to wait.

An information providing apparatus according to an aspect of the present disclosure includes: an identifying means for identifying a customer; an acquiring means for acquiring information regarding a prescription issued to the identified customer and information indicating a work status of medicine dispensing based on the prescription; a predicting means for predicting a time for the customer to wait based on at least one of the information regarding the prescription and the information indicating the work status of medicine dispensing based on the prescription; and an outputting means for outputting information to be provided to the customer in a case where the predicted time for the customer to wait exceeds a threshold value.

An information providing method according to the aspect of the present disclosure causes a computer to: identify a customer; acquire information regarding a prescription issued to the identified customer and information indicating a work status of medicine dispensing based on the prescription; predict a time for the customer to wait based on at least one of the information regarding the prescription and the information indicating the work status of medicine dispensing based on the prescription; and output information to be provided to the customer in a case where the predicted time for the customer to wait exceeds a threshold value.

A program according to the aspect of the present disclosure causes a computer to execute: processing for identifying a customer; processing for acquiring information regarding a prescription issued to the identified customer and information indicating a work status of medicine dispensing based on the prescription; processing for predicting a time for the customer to wait based on at least one of the information regarding the prescription and the information indicating the work status of medicine dispensing based on the prescription; and processing for outputting information to be provided to the customer in a case where the predicted time for the customer to wait exceeds a threshold value.

### BRIEF DESCRIPTION OF THE DRAWINGS

Exemplary features and advantages of the present disclosure will become apparent from the following detailed description when taken with the accompanying drawings in which:

FIG. 1 is a diagram roughly illustrating an example of an information providing system according to an example embodiment;

FIG. 2 is a block diagram illustrating a configuration of an information providing apparatus according to the example embodiment;

FIG. 3 is a flowchart illustrating a flow of operation in the information providing apparatus according to the example embodiment;

FIG. 4 is a diagram roughly illustrating an example of an information providing system according to an example embodiment;

FIG. 5 is a block diagram illustrating a configuration of an information providing apparatus according to the example embodiment;

FIG. 6 is a flowchart illustrating a flow of operation in the information providing apparatus according to the example embodiment;

FIG. 7 is a diagram roughly illustrating an example of an information providing system according to an example embodiment;

FIG. 8 is a block diagram illustrating a configuration of an information providing apparatus according to the example embodiment;

FIG. 9 is a flowchart illustrating a flow of operation in the information providing apparatus according to the example embodiment; and

FIG. 10 is a block diagram illustrating a hardware configuration of the information providing apparatus according to one of the example embodiments.

### EXAMPLE EMBODIMENT

Some example embodiments of the present disclosure will now be described herein with reference to the accompanying drawings.

### First Example Embodiment

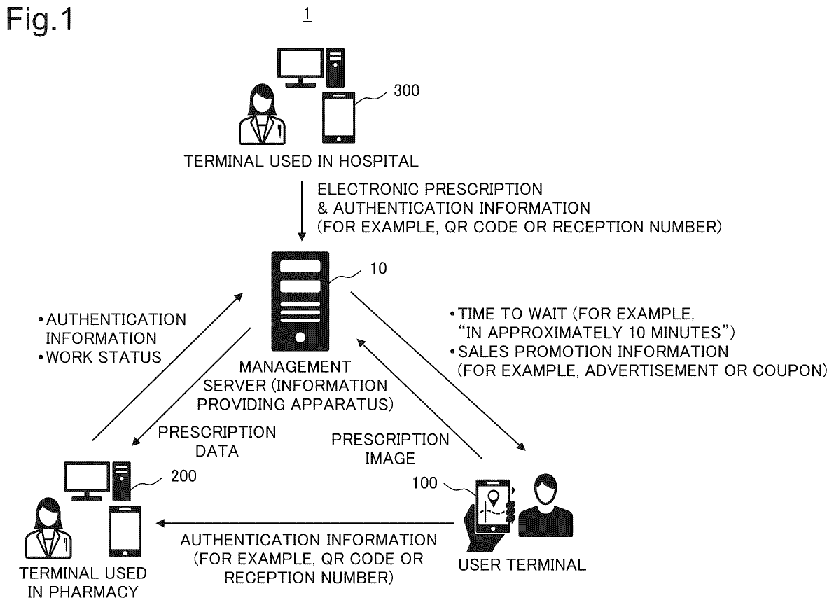

A first example embodiment of the present disclosure will now be described herein with reference to FIGS. 1 to 3.

### (Information Providing System 1)

FIG. 1 is a diagram roughly illustrating an example of an information providing system 1 according to an example embodiment. As illustrated in FIG. 1, the information providing system 1 includes a user terminal 100, a terminal 200 used in a pharmacy, a terminal 300 used in a hospital, and an information providing apparatus 10.

The user terminal 100 is a communication device (for example, a smartphone or a tablet terminal) possessed by a customer visiting a dispensing corner in a pharmacy, a drug store, or a store such as a supermarket. In the user terminal 100, an application for accessing a management server (hereinafter referred to as the information providing apparatus 10) may be installed in advance. Although a case where a user visits a certain store in which a pharmacy is available will be described herein as an example, it is possible to read a “pharmacy” in the description as a “drug store” or a “dispensing corner” in a replaced manner.

The customer visits the store in which the pharmacy is available and presents authentication information received by the user terminal 100 to a staff in the pharmacy.

The terminal 200 used in the pharmacy is a communication terminal used by the staff in the pharmacy. The terminal 200 used in the pharmacy is communicably coupled to the information providing apparatus 10 via a system (not illustrated) in the pharmacy.

The staff in the pharmacy inputs the authentication information (to be described later) presented by the customer into the terminal 200 used in the pharmacy. The terminal 200 used in the pharmacy transmits the inputted authentication information to the information providing apparatus 10. Then, the terminal 200 used in the pharmacy receives prescription data from the information providing apparatus 10. The prescription data includes information indicating content of a prescription.

The staff in the pharmacy inputs information (for example, a stage of progress of medicine dispensing) indicating a work status of medicine dispensing based on the prescription to the terminal 200 used in the pharmacy. The terminal 200 used in the pharmacy transmits the information indicating the working status of medicine dispensing based on the prescription to the information providing apparatus 10.

The terminal 300 used in the hospital is communicably coupled to the information providing apparatus 10 via a system (not illustrated) in the hospital. The terminal 300 used in the hospital issues an electronic prescription based on a result of diagnosis on a patient (a person who is the customer described above). The electronic prescription includes information identifying the patient (for example, a name, a gender, an insurance identification number, and a personal number called my number), in addition to information regarding the prescription.

The terminal 300 used in the hospital issues authentication information (for example, a two-dimensional code such as a quick response (QR) code (registered trademark), a bar code, or a reception number) associated with the electronic prescription.

Then, the terminal 300 used in the hospital transmits the issued authentication information to the information providing apparatus 10 via the system in the hospital. The terminal 300 used in the hospital further transmits the authentication information to the user terminal 100. The terminal 300 used in the hospital may further transmit the electronic prescription together with the authentication information to the information providing apparatus 10. Alternatively, the terminal 300 used in the hospital May transmit the electronic prescription associated with the authentication information to the information providing apparatus 10 in response to a request from the information providing apparatus 10.

It is also possible to use biological information of the customer as authentication information of the customer. In this case, the terminal 300 used in the hospital collects, by using a desired means, biological information (for example, an image of a face, an image of a fingerprint, an image of an iris, or other biological data) of the customer, and transmits the collected biological information (an example of authentication information) to the information providing apparatus 10. One that issues authentication information is not limited to the terminal 300 used in the hospital. The user terminal 100 may issue authentication information and transmit the issued authentication information to the information providing apparatus 10.

The information providing apparatus 10 identifies the customer and acquires information regarding the prescription issued to the identified customer.

In one example, the information providing apparatus 10 acquires the electronic prescription associated with the authentication information received from the terminal 200 used in the pharmacy from the terminal 300 used in the hospital. The information providing apparatus 10 extracts information for identifying the customer from the electronic prescription.

The information providing apparatus 10 further acquires information indicating a work status of medicine dispensing based on the prescription. In one example, the information providing apparatus 10 receives information indicating a stage of progress of medicine dispensing (for example, “not started”, “started”, or “completed”) from the terminal 200 used in the pharmacy. Alternatively, the information providing apparatus 10 may create information indicating a work status of medicine dispensing based on a prescription based on an image of a situation in a store, which is captured by a camera installed in a pharmacy. For example, the information providing apparatus 10 determines whether there is a pharmacist who is available for preparing a medicine by analyzing the image of the situation in the store, and creates information indicating a work status of medicine dispensing based on the prescription based on a result of the determination.

The information providing apparatus 10 predicts a time for the customer to wait based on at least one of the information regarding the prescription and the information indicating the work status of medicine dispensing based on the prescription. Then, when the predicted time for the customer to wait exceeds a threshold value, the information providing apparatus 10 outputs information to be provided to the customer. A method for determining the threshold value is not particularly limited. In one example, the threshold value is five minutes or 10 minutes.

In one example, the information providing apparatus 10 transmits sales promotion information (for example, an advertisement or a coupon) to the user terminal 100. In another example, the information providing apparatus 10 transmits information indicating a time to wait (for example, “in 10 minutes”) to the user terminal 100.

A configuration and operation of the information providing apparatus 10 according to the example embodiment will now be described herein.

### (Configuration of Information Providing Apparatus 10)

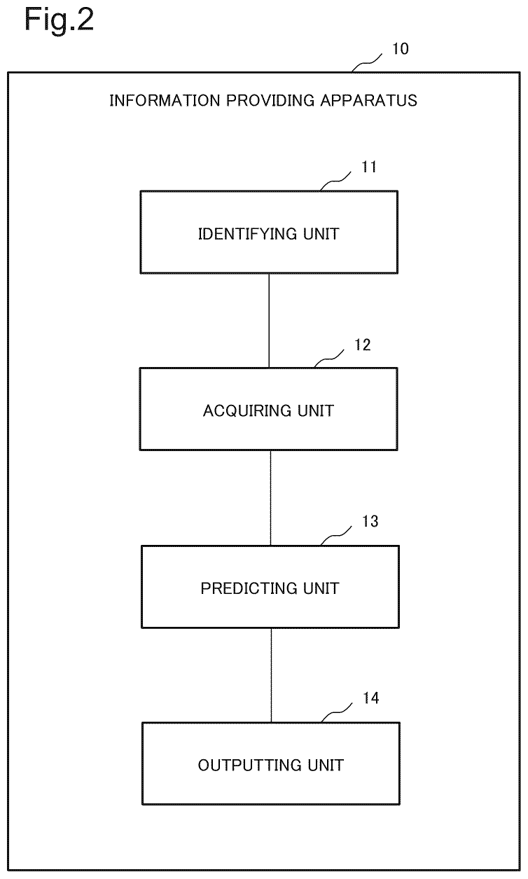

The configuration of the information providing apparatus 10 according to the example embodiment will now be described herein with reference to FIG. 2. FIG. 2 is a block diagram illustrating the configuration of the information providing apparatus 10.

As illustrated in FIG. 2, the information providing apparatus 10 includes an identifying unit 11, an acquiring unit 12, a predicting unit 13, and an outputting unit 14.

The identifying unit 11 identifies a customer. The identifying unit 11 is an example of an identifying means. For example, the identifying unit 11 acquires authentication information issued together with an electronic prescription by the terminal 300 used in the hospital from the terminal 200 used in the pharmacy or the user terminal 100.

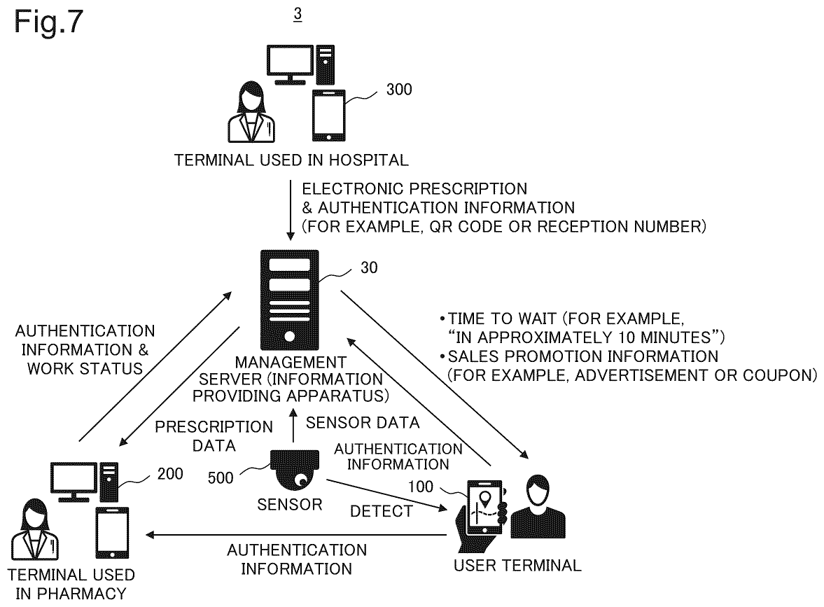

In another example, the identifying unit 11 acquires biological information of the customer visiting the pharmacy from a sensor 500 (FIG. 7) installed near an entrance of the pharmacy. An installation position of the sensor 500 is not particularly limited to the entrance of the pharmacy, and may be the entrance of a store.

The identifying unit 11 outputs information (for example, the authentication information or the biological information) indicating the identified customer to the acquiring unit 12.

The acquiring unit 12 acquires information regarding the prescription issued to the identified customer. The acquiring unit 12 is an example of an acquiring means.

For example, the acquiring unit 12 receives the authentication information or the biological information as information indicating the identified customer from the identifying unit 11. The acquiring unit 12 acquires, by using the authentication information or the biological information, the electronic prescription (an example of information regarding the prescription) associated with the authentication information or the biological information from the terminal 300 used in the hospital.

In another example, the acquiring unit 12 receives an image (hereinafter referred to as a “prescription image”) acquired by photographing the prescription in a form of a piece of paper (an example of information regarding the prescription) from the user terminal 100. Alternatively, the acquiring unit 12 may create information indicating a work status of medicine dispensing based on the prescription based on an image of a situation in the store, which is captured by the camera installed in the pharmacy. For example, the acquiring unit 12 analyzes the image of the situation in the store to determine whether there is a pharmacist who is available for preparing a medicine, and creates information indicating a work status of medicine dispensing based on the prescription based on a result of the determination.

It is possible to utilize an application installed in the user terminal 100 to transmit the prescription image from the user terminal 100 to the information providing apparatus 10. Alternatively, an email attached with the prescription image may be transmitted from the user terminal 100 to the information providing apparatus 10.

The acquiring unit 12 outputs information (for example, the electronic prescription or the prescription image) regarding the prescription issued to the identified customer to the predicting unit 13.

The predicting unit 13 predicts a time for the customer to wait based on at least one of the information regarding the prescription and the information indicating the work status of medicine dispensing based on the prescription. The predicting unit 13 is an example of a predicting means.

For example, the predicting unit 13 receives the prescription image or the electronic prescription as information regarding the prescription from the acquiring unit 12. Then, the predicting unit 13 predicts a time for the customer to wait by using the information regarding the prescription (for example, the prescription image or the electronic prescription). For example, the predicting unit 13 predicts a time for the customer to wait based on a type or an amount of a medicine included in the prescription. In another example, the predicting unit 13 predicts a time for the customer to wait based on presence or absence of a step that takes a more period of time (for example, a step of mixing a medicine in a form of powder, syrup, or ointment, or a step of packaging a medicine). For such a step that takes a more period of time, a guide for a period of time required for the step may be defined. In a case where there is a step that takes a more period of time, the predicting unit 13 may utilize this guide period of time to predict a time for the customer to wait.

In another example, the predicting unit 13 receives information indicating a stage of progress of medicine dispensing from the terminal 200 used in the pharmacy as information indicating a work status of medicine dispensing based on the prescription. Then, the predicting unit 13 predicts a time for the customer to wait by using the information indicating the work status of medicine dispensing.

The predicting unit 13 outputs information indicating the predicted time for the customer to wait to the outputting unit 14.

The outputting unit 14 receives the information indicating the predicted time for the customer to wait from the predicting unit 13. The outputting unit 14 determines whether the predicted time for the customer to wait exceeds a threshold value. When the predicted time for the customer to wait exceeds the threshold value (for example, five minutes or 10 minutes), the outputting unit 14 outputs information to be provided to the customer. The outputting unit 14 is an example of an outputting means.

For example, the outputting unit 14 transmits sales promotion information (for example, an advertisement or a coupon) and/or information indicating a time for the customer to wait (for example, “in 10 minutes”) to the user terminal 100. In another example, the outputting unit 14 causes a signage or a monitor (including a television image receiver) installed in the store to display sales promotion information or information indicating a time to wait. Performing sales promotion of a product suitable for the customer during the time to wait in this way makes it possible to improve sales in the store.

The outputting unit 14 may determine content of information to be provided to the customer in accordance with a length of a predicted time to wait or a type of a medicine included in the prescription medicine.

For example, when the predicted time to wait is equal to or more than five minutes and less than 10 minutes, the outputting unit 14 transmits a coupon to the user terminal 100. Alternatively, the outputting unit 14 is able to change a discount rate (a degree of discount) of a coupon in accordance with a predicted time to wait. When a predicted time to wait is equal to or more than 10 minutes, on the other hand, the outputting unit 14 transmits an advertisement or a coupon for a reading to the user terminal 100.

In another example, the outputting unit 14 estimates a physical condition or a symptom of the customer in accordance with a type of a medicine included in the prescription medicine, and transmits an advertisement of a product suitable for the physical condition or the symptom of the customer (for example, having cough and/or fever) to the user terminal 100. It is also possible to estimate the physical condition of the customer based on the biological information (for example, an image of the face) of the customer (a third example embodiment).

A method for allowing the outputting unit 14 to determine content of information to be provided to the customer in accordance with a length of a predicted time to wait or a type of a medicine included in a prescription medicine is not limited to the specific example described in here. In addition to these examples, the outputting unit 14 is able to determine content of information to be provided to the customer in various modes.

### (Operation in Information Providing Apparatus 10)

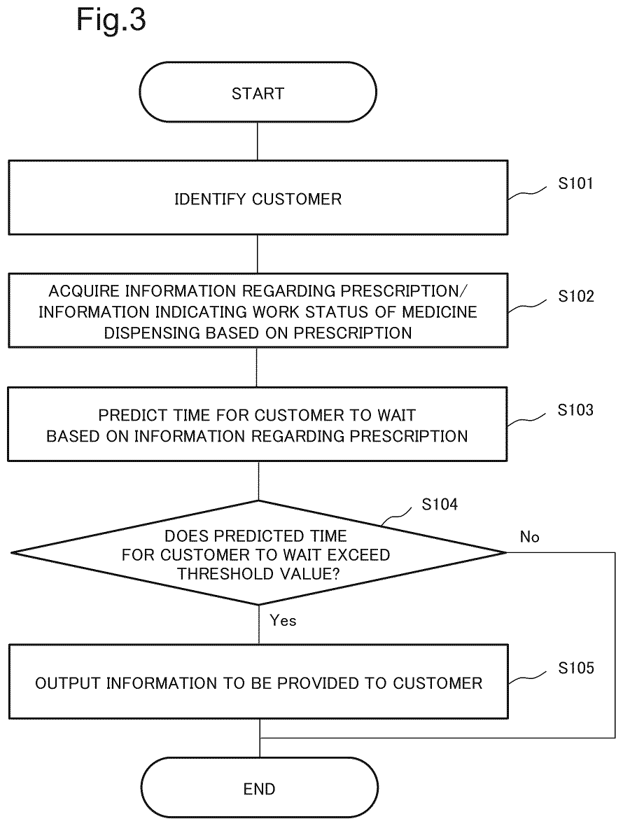

Operation in the information providing apparatus 10 according to the example embodiment will now be described herein with reference to FIG. 3. FIG. 3 is a flowchart illustrating a flow of operation in the information providing apparatus 10.

The identifying unit 11 identifies a customer (S101). The identifying unit 11 outputs information indicating the identified customer to the acquiring unit 12.

The acquiring unit 12 acquires information regarding a prescription issued to the identified customer and information indicating a work status of medicine dispensing based on the prescription (S102). The acquiring unit 12 outputs pieces of the acquired information to the predicting unit 13.

The predicting unit 13 predicts a time for the customer to wait based on at least one of the biological information of the customer and the information regarding the prescription (S103). The predicting unit 13 outputs information indicating the predicted time for the customer to wait to the outputting unit 14.

The outputting unit 14 determines whether the predicted time for the customer to wait exceeds a threshold value (S104). When the predicted time for the customer to wait exceeds the threshold value (Yes at S104), the outputting unit 14 outputs information to be provided to the customer (S105).

When the predicted time for the customer to wait is equal to or less than the threshold value (No at S104), on the other hand, the outputting unit 14 does not output information to be provided to the customer.

As described above, operation in the information providing apparatus 10 according to the example embodiment ends.

### (Effects of Present Example Embodiment)

According to the configuration of the present example embodiment, the identifying unit 11 identifies a customer. The acquiring unit 12 acquires information regarding a prescription issued to the identified customer and information indicating a work status of medicine dispensing based on the prescription. The predicting unit 13 predicts a time for the customer to wait based on at least one of the information regarding the prescription and the information indicating the work status of medicine dispensing based on the prescription. When the predicted time for the customer to wait exceeds the threshold value, the outputting unit 14 outputs information to be provided to the customer.

In this way, it is possible to provide, when a predicted time for the customer to wait exceeds a threshold value, information for using the time to wait to the customer waiting for delivery of a prescription medicine.

### Second Example Embodiment

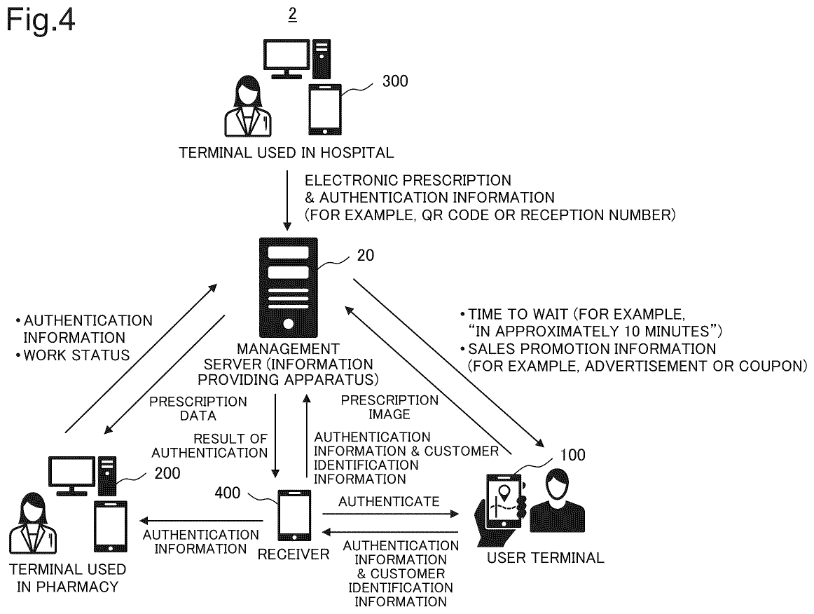

A second example embodiment of the present disclosure will now be described herein with reference to FIGS. 4 to 6. In the second example embodiment, a configuration for identifying a customer by using customer identification information after authenticating the customer will be described.

In the second example embodiment, identical reference numerals to the reference numerals in the first example embodiment are given to components common to the components in the first example embodiment, and description of the components will be omitted.

### (Information Providing System 2)

FIG. 4 is a diagram roughly illustrating an example of an information providing system 2 according to an example embodiment; As illustrated in FIG. 4, the information providing system 2 includes the user terminal 100, the terminal 200 used in the pharmacy, the terminal 300 used in the hospital, and an information providing apparatus 20. The information providing system 2 further includes a receiver 400.

The receiver 400 authenticates a customer visiting the pharmacy. For example, the receiver 400 acquires a QR code or a reception number issued by the terminal 300 used in the hospital or biological information (an example of authentication information) of the customer. The receiver 400 receives information for identifying the customer (for example, a personal number called my number or an insurance identification number) (hereinafter referred to as “customer identification information”) from the customer.

The receiver 400 transmits the authentication information acquired from the customer and the inputted customer identification information to the information providing apparatus 20, and requests the information providing apparatus 20 for authenticating the customer. The receiver 400 receives a result of the authentication for the customer from the information providing apparatus 20. Alternatively, the receiver 400 may authenticate the customer, in replacement of the information providing apparatus 20. In the present example, the receiver 400 authenticates the customer by acquiring authentication information associated with an electronic prescription from the information providing apparatus 20 and collating the authentication information acquired from the customer with the authentication information acquired from the information providing apparatus 20.

When the information providing apparatus 20 (or the receiver 400) is successful in authenticating the customer, the receiver 400 transmits the authentication information acquired from the customer to the terminal 200 used in the pharmacy.

The terminal 200 used in the pharmacy transmits the authentication information received from the receiver 400 to the information providing apparatus 20. Then, the terminal 200 used in the pharmacy receives prescription data from the information providing apparatus 20.

The information providing apparatus 20 according to the example embodiment identifies the customer and acquires information regarding the prescription issued to the identified customer.

In one example, the information providing apparatus 20 acquires an electronic prescription associated with authentication information received from the terminal 200 used in the pharmacy from the terminal 300 used in the hospital. The information providing apparatus 20 extracts information for identifying the customer from the electronic prescription.

The information providing apparatus 20 further acquires information indicating a work status of medicine dispensing based on the prescription. In one example, the information providing apparatus 20 receives information indicating a stage of progress of medicine dispensing (for example, “not started”, “started”, or “completed”) from the terminal 200 used in the pharmacy.

The information providing apparatus 20 predicts a time for the customer to wait based on at least one of the information regarding the prescription and the information indicating the work status of medicine dispensing based on the prescription. Then, when the predicted time for the customer to wait exceeds a threshold value, the information providing apparatus 20 outputs information to be provided to the customer.

In one example, the information providing apparatus 20 transmits sales promotion information (for example, an advertisement or a coupon) to the user terminal 100. In another example, the information providing apparatus 20 transmits information indicating a time to wait (for example, “in 10 minutes”) to the user terminal 100.

A configuration and operation of the information providing apparatus 20 according to the example embodiment will now be described herein.

### (Configuration of Information Providing Apparatus 20)

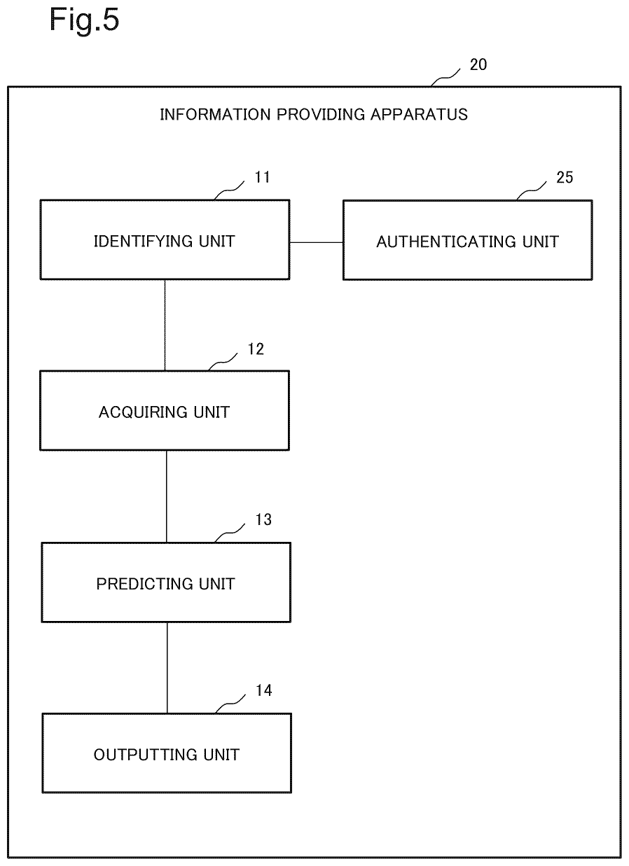

The configuration of the information providing apparatus 20 according to the example embodiment will now be described herein with reference to FIG. 5. FIG. 5 is a block diagram illustrating the configuration of the information providing apparatus 20.

As illustrated in FIG. 5, the information providing apparatus 20 includes the identifying unit 11, the acquiring unit 12, the predicting unit 13, and the outputting unit 14. The information providing apparatus 20 further includes an authenticating unit 25.

The authenticating unit 25 authenticates the customer by using authentication information issued based on information regarding a prescription. The authenticating unit 25 is an example of an authenticating means.

For example, the authenticating unit 25 receives authentication information inputted by the customer and identification information of the customer from the receiver 400 (FIG. 4). The authenticating unit 25 authenticates the customer by using the authentication information associated with the identification information of the customer and collating the authentication information with the authentication information inputted by the customer.

After that, the authenticating unit 25 transmits a result of the authentication (indicating whether the authentication of the customer is successful or failed) of the customer to the receiver 400.

### (Operation in Information Providing Apparatus 20)

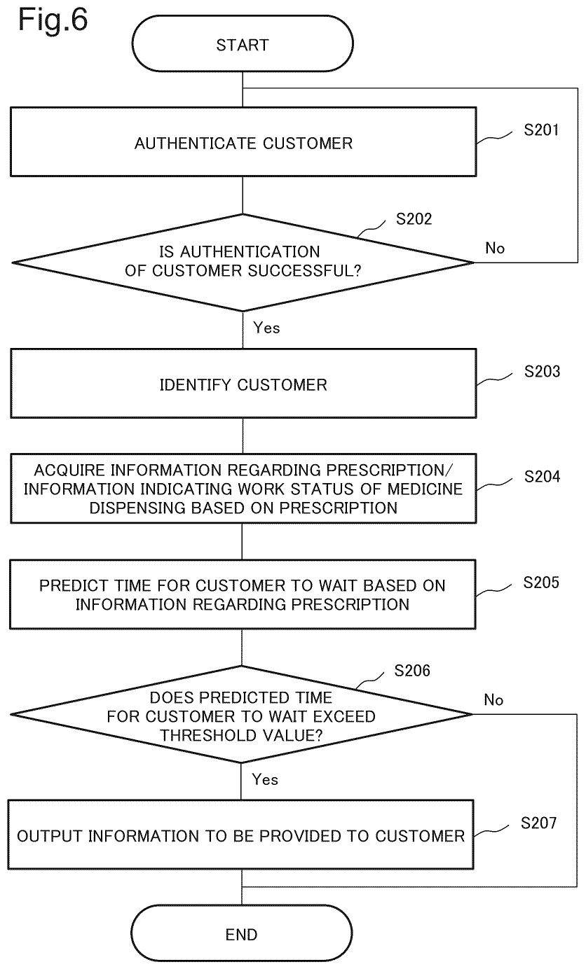

Operation in the information providing apparatus 20 according to the example embodiment will now be described herein with reference to FIG. 6. FIG. 6 is a flowchart illustrating a flow of operation in the information providing apparatus 20.

The authenticating unit 25 authenticates a customer by using authentication information issued based on information regarding a prescription (S201). The authenticating unit 25 transmits a result of the authentication of the customer to the identifying unit 11.

The identifying unit 11 determines whether the authentication of the customer is successful (S202). When the authentication of the customer is failed (No at S202), the flow returns to step S201.

When the authentication of the customer is successful (Yes at S202), on the other hand, the identifying unit 11 identifies the customer (S203). The identifying unit 11 outputs information indicating the identified customer to the acquiring unit 12.

The acquiring unit 12 acquires the information regarding the prescription issued to the identified customer and information indicating a work status of medicine dispensing based on the prescription (S204). The acquiring unit 12 outputs pieces of the acquired information to the predicting unit 13.

The predicting unit 13 predicts a time for the customer to wait based on at least one of the biological information of the customer and the information regarding the prescription (S205). The predicting unit 13 outputs information indicating the predicted time for the customer to wait to the outputting unit 14.

The outputting unit 14 determines whether the predicted time for the customer to wait exceeds a threshold value (S206). When the predicted time for the customer to wait exceeds the threshold value (Yes at S206), the outputting unit 14 outputs information to be provided to the customer (S207).

When the predicted time for the customer to wait is equal to or less than the threshold value (No at S206), on the other hand, the outputting unit 14 does not output information to be provided to the customer.

As described above, operation in the information providing apparatus 20 according to the example embodiment ends.

### (Effects of Present Example Embodiment)

According to the configuration of the present example embodiment, the identifying unit 11 identifies a customer. The acquiring unit 12 acquires information regarding a prescription issued to the identified customer and information indicating a work status of medicine dispensing based on the prescription. The predicting unit 13 predicts a time for the customer to wait based on at least one of the information regarding the prescription and the information indicating the work status of medicine dispensing based on the prescription. When the predicted time for the customer to wait exceeds the threshold value, the outputting unit 14 outputs information to be provided to the customer.

In this way, it is possible to provide, when a predicted time for the customer to wait exceeds a threshold value, information for using the time to wait to the customer waiting for delivery of a prescription medicine.

According to the configuration of the present example embodiment, the authenticating unit 25 authenticates a customer by using authentication information issued based on information regarding a prescription. As a result, it is possible to prevent a prescription medicine from being delivered or sales promotion information or an advertisement from being provided to a wrong customer.

### Third Example Embodiment

A third example embodiment of the present disclosure will now be described herein with reference to FIGS. 7 to 9. In the third example embodiment, a configuration in which a physical condition of a customer is estimated, and information for using a time to wait is not provided to the customer with a bad physical condition will be described.

In the third example embodiment, identical reference numerals to the reference numerals in the first or second example embodiment are given to components common to the components in the first or second example embodiment, and description of the components will be omitted.

### (Information Providing System 3)

FIG. 7 is a diagram roughly illustrating an example of an information providing system 3 according to an example embodiment; As illustrated in FIG. 7, the information providing system 3 includes the user terminal 100, the terminal 200 used in the pharmacy, the terminal 300 used in the hospital, and an information providing apparatus 30. The information providing system 3 further includes the sensor 500.

The sensor 500 detects a customer visiting a store in which a pharmacy is available. For example, the sensor 500 is a capacitance sensor, an optical (image) sensor, an infrared sensor, or an ultrasonic sensor. In another example, the sensor 500 is a beacon (a radio wave sensor). In the case of this example, the sensor 500 detects a radio wave that the user terminal 100 transmits. The sensor 500 may be provided in the receiver 400 (FIG. 4) described in the second example embodiment.

The sensor 500 collects biological information (for example, an image of a face, an image of a fingerprint, an image of an iris, or other biological data) of the customer. The sensor 500 transmits the collected biological information (sensor data) of the customer to the information providing apparatus 30.

The information providing apparatus 30 according to the example embodiment identifies the customer and acquires information regarding a prescription issued to the identified customer.

In one example, the information providing apparatus 30 identifies the customer based on the biological information (an example of customer identification information) received from the sensor 500. Then, the information providing apparatus 30 acquires an electronic prescription associated with the customer identification information from the terminal 300 used in the hospital. The information providing apparatus 30 extracts information for identifying the customer from the electronic prescription.

The information providing apparatus 30 further acquires information indicating a work status of medicine dispensing based on the prescription. In one example, the information providing apparatus 30 receives information indicating a stage of progress of medicine dispensing (for example, “not started”, “started”, or “completed”) from the terminal 200 used in the pharmacy.

The information providing apparatus 30 estimates a physical condition of the customer based on at least one of the biological information of the customer and the information regarding the prescription.

The information providing apparatus 30 predicts a time for the customer to wait based on at least one of the information regarding the prescription and the information indicating the work status of medicine dispensing based on the prescription. Then, when the predicted time for the customer to wait exceeds a threshold value, the information providing apparatus 30 outputs information to be provided to the customer.

In one example, the information providing apparatus 30 transmits sales promotion information (for example, an advertisement or a coupon) to the user terminal 100. In another example, the information providing apparatus 30 transmits information indicating a time to wait (for example, “in 10 minutes”) to the user terminal 100.

However, when the customer is estimated to be in a bad (or unhealthy) physical condition, the information providing apparatus 30 does not output information to be provided to the customer even when a predicted time for the customer to wait exceeds the threshold value.

A configuration and operation of the information providing apparatus 30 according to the example embodiment will now be described herein.

### (Configuration of Information Providing Apparatus 30)

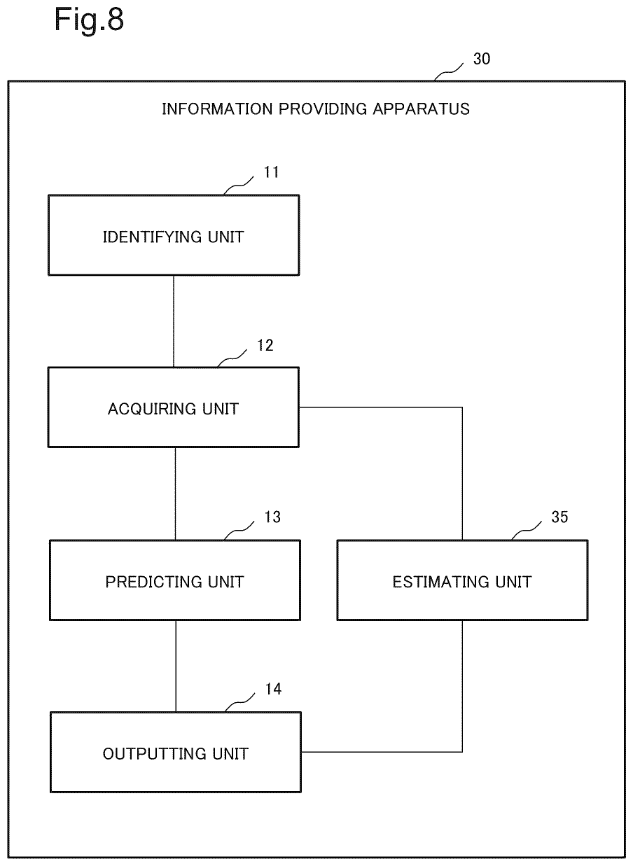

The configuration of the information providing apparatus 30 according to the example embodiment will now be described herein with reference to FIG. 8. FIG. 8 is a block diagram illustrating the configuration of the information providing apparatus 30.

As illustrated in FIG. 8, the information providing apparatus 30 includes the identifying unit 11, the acquiring unit 12, the predicting unit 13, and the outputting unit 14. The information providing apparatus 30 further includes an estimating unit 35.

The estimating unit 35 estimates a physical condition of a customer based on at least one of biological information of the customer and information regarding a prescription. The estimating unit 35 is an example of an estimating means.

For example, the estimating unit 35 receives, from the sensor 500, biological information of the customer (for example, an image of a face, an image of a fingerprint, an image of an iris, or other biological data) as sensing data collected from the customer. The estimating unit 35 estimates a physical condition of the customer by using the biological information of the customer. In the present example, the estimating unit 35 is able to use, to estimate a physical condition of the customer, an estimator acquired by performing learning of a relationship between biological information of the customer and a determination of whether a physical condition of the customer is good or bad.

For example, the estimating unit 35 estimates a physical condition of the customer by analyzing complexion, movement of the face, a line of sight, blinking, a diameter of a pupil, movement of an iris, and facial expression of the customer, for example, which are included in a photographed image of the face of the customer.

Alternatively, the estimating unit 35 may estimate a physical condition of the customer by estimating emotion of the customer based on a motion of a body of the customer, which is acquired from a captured image of the customer.

Alternatively, the estimating unit 35 may estimate a physical condition of the customer based on a temperature of the body of the customer, which is detected by an infrared sensor (an example of the sensor 500). For example, the estimating unit 35 estimates that a physical condition of the customer is bad when the temperature of the body of the customer is equal to or more than a threshold value.

In another example, the estimating unit 35 acquires information regarding a prescription issued to the customer from the acquiring unit 12. The estimating unit 35 estimates a physical condition of the customer based on the prescription issued to the customer. In the present example, the estimating unit 35 is able to use a table indicating a correspondence relationship between a prescription medicine indicated in a prescription or a type of a medicine included in the prescription medicine and a physical condition of the customer.

### (Operation in Information Providing Apparatus 30)

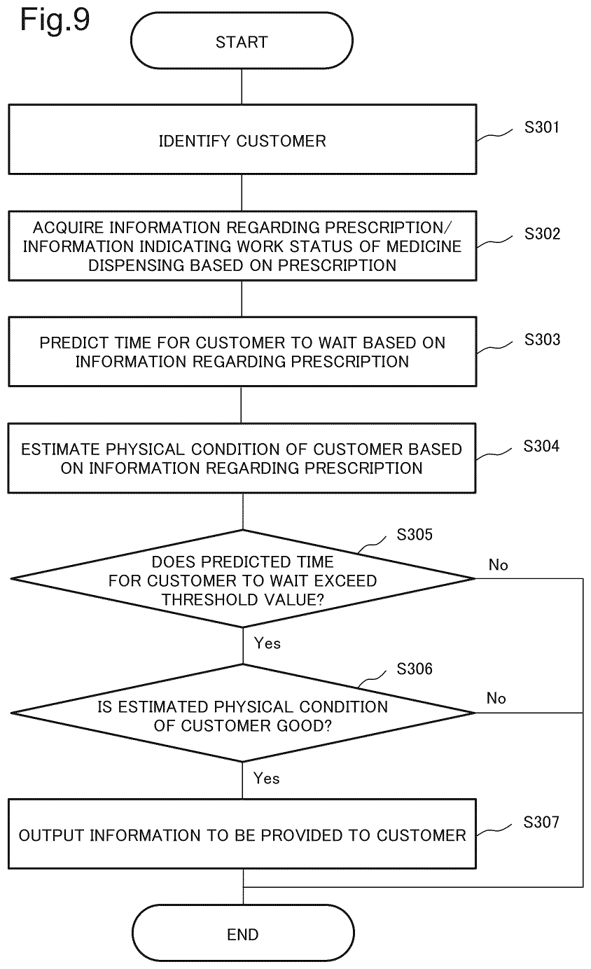

Operation in the information providing apparatus 30 according to the example embodiment will now be described herein with reference to FIG. 9. FIG. 9 is a flowchart illustrating a flow of operation in the information providing apparatus 30.

The identifying unit 11 identifies a customer (S301). The identifying unit 11 outputs information indicating the identified customer to the acquiring unit 12.

The acquiring unit 12 acquires information regarding a prescription issued to the identified customer and information indicating a work status of medicine dispensing based on the prescription (S302). The acquiring unit 12 outputs pieces of the acquired information to the predicting unit 13 and the estimating unit 35.

The predicting unit 13 predicts a time for the customer to wait based on at least one of the biological information of the customer and the information regarding the prescription (S303). The predicting unit 13 outputs information indicating the predicted time for the customer to wait to the outputting unit 14.

The estimating unit 35 estimates a physical condition of the customer based on at least one of the biological information of the customer and the information regarding the prescription (S304).

The outputting unit 14 determines whether the predicted time for the customer to wait exceeds a threshold value (S305).

When the predicted time for the customer to wait exceeds the threshold value (Yes at S305), the outputting unit 14 further determines whether the estimated physical condition of the customer is good (S306).

When the estimated physical condition of the customer is good (Yes at S306), the outputting unit 14 outputs information to be provided to the customer (S307).

When the predicted time for the customer to wait is equal to or less than the threshold value (No at S305) or when the estimated physical condition of the customer is not good (No at S306), on the other hand, the outputting unit 14 does not output information to be provided to the customer.

As described above, operation in the information providing apparatus 30 according to the example embodiment ends.

### (Effects of Present Example Embodiment)

According to the configuration of the present example embodiment, the identifying unit 11 identifies a customer. The acquiring unit 12 acquires information regarding a prescription issued to the identified customer and information indicating a work status of medicine dispensing based on the prescription. The predicting unit 13 predicts a time for the customer to wait based on at least one of the information regarding the prescription and the information indicating the work status of medicine dispensing based on the prescription. When the predicted time for the customer to wait exceeds the threshold value, the outputting unit 14 outputs information to be provided to the customer.

In this way, it is possible to provide, when a predicted time for the customer to wait exceeds a threshold value, information for using the time to wait to the customer waiting for delivery of a prescription medicine.

According to the configuration of the present example embodiment, the estimating unit 35 estimates a physical condition of the customer based on at least one of the biological information of the customer and the information regarding the prescription. When the estimated physical condition of the customer is bad (or not good), information to be provided to the customer is not outputted even when a predicted time for the customer to wait exceeds the threshold value. As a result, unnecessary information is not provided to the customer in a bad physical condition, preventing the customer from being annoyed.

### (Hardware Configuration)

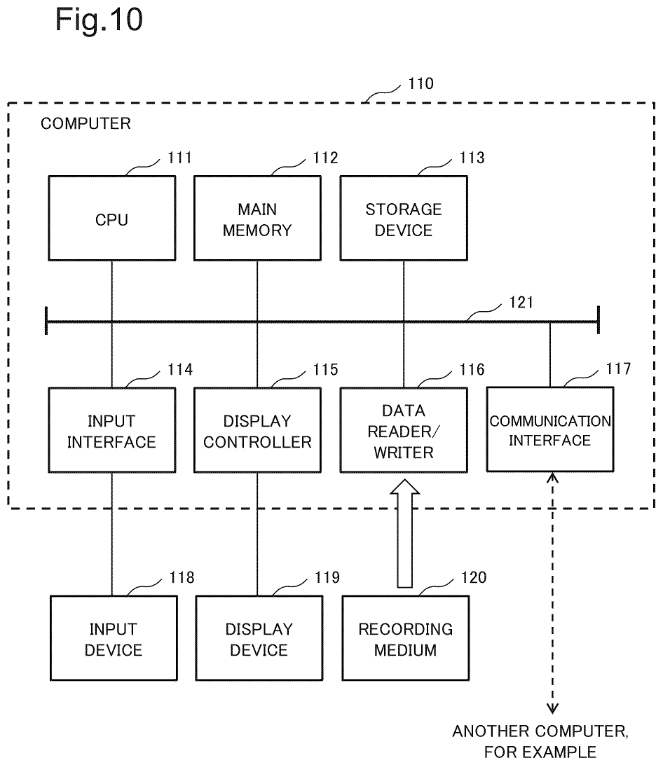

Each of the components in the information providing apparatuses 10, 20, and 30 described in the first to third example embodiments represents a block in functional unit. Some or all of these components are achieved by, for example, an information processing apparatus as illustrated in FIG. 10. FIG. 10 is a block diagram illustrating an example of a hardware configuration of the information processing apparatus.

As illustrated in FIG. 10, a computer 110 includes a central processing unit (CPU) 111, a main memory 112, a storage device 113, an input interface 114, a display controller 115, a data reader/writer 116, and a communication interface 117. These components are coupled to each other via a bus 121, making it possible to perform data communication with each other. The computer 110 may include a graphics processing unit (GPU) or a field-programmable gate array (FPGA), in addition to the CPU 111 or instead of the CPU 111.

The CPU 111 deploys, in the main memory 112, programs (codes) according to the present example embodiment, which are stored in the storage device 113, and executes the programs in a predetermined order to perform various types of operation. The main memory 112 is, typically, a volatile storage device such as a dynamic random access memory (DRAM). The programs according to the present example embodiment are provided in a state of being stored in a computer-readable recording medium 120. The programs according to the present example embodiment may be distributed on the Internet coupled via the communication interface 117.

Specific examples of the storage device 113 includes a hard disk drive and a semiconductor storage device such as a flash memory. The input interface 114 mediates data transmission between the CPU 111 and an input device 118 including a keyboard and a mouse, for example. The display controller 115 is coupled to a display device 119, and controls displaying on the display device 119.

The data reader/writer 116 mediates data transmission between the CPU 111 and the recording medium 120, and reads a program from the recording medium 120 and writes a result of processing in the computer 110 into the recording medium 120. The communication interface 117 mediates data transmission between the CPU 111 and another computer.

One specific example of the recording medium 120 is a general-purpose semiconductor storage device such as a compact flash (CF (registered trademark)) or a secure digital (SD), a magnetic recording medium such as a flexible disk, or an optical recording medium such as a compact disk read only memory (CD-ROM).

### (Related Art)

The technique related to the present disclosure provides sales promotion information such as product recommendations to a customer in accordance with information such as an attribute and a purchase record of the customer.

When a patient has arrived a dispensing pharmacy, there may be a case where a prescription medicine has not yet dispensed. In that case, the patient needs to use a time to wait at the pharmacy until the prescription medicine to be delivered is prepared. However, the patient may not be sure how to use the time to wait.

According to the aspect of the present disclosure, it is possible to provide information for using a time to wait to a customer waiting for delivery of a prescription medicine.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

It will be understood by those of ordinary skill in the art that various changes in form and details may be made therein without departing from the spirit and scope of the present disclosure as defined by the claims.

Although a plurality of types of operation are described in order each in a form of flowchart, the order of those described does not limit the order of executing the plurality of types of operation. Therefore, when each of the example embodiments is to be implemented, it may be possible to change the order of the plurality of types of operation within a range where there will be no interference in content.

Further, it is noted that the inventor's intent is to retain all equivalents of the claimed disclosure even if the claims are amended during prosecution.

### SUPPLEMENTARY NOTE

Although some or all of the example embodiments described above may be described as the following supplementary notes, the present disclosure is not limited to those described below.

### Supplementary Note 1

An information providing apparatus includes:
an identifying means for identifying a customer;
an acquiring means for acquiring information regarding a prescription issued to the identified customer and information indicating a work status of medicine dispensing based on the prescription;
a predicting means for predicting a time for the customer to wait based on at least one of the information regarding the prescription and the information indicating the work status of medicine dispensing based on the prescription; and
an outputting means for outputting information to be provided to the customer when the predicted time for the customer to wait exceeds a threshold value.

### Supplementary Note 2

The information providing apparatus according to Supplementary Note 1, in which
the identifying means identifies the customer by using biological information of the customer.

### Supplementary Note 3

The information providing apparatus according to Supplementary Note 1 or 2, in which
the acquiring means acquires the information regarding the prescription from an electronic prescription corresponding to the prescription or image data acquired by photographing the prescription.

### Supplementary Note 4

The information providing apparatus according to Supplementary Note 1 or 2, in which
the acquiring means acquires the information indicating the working status of medicine dispensing based on the prescription from a terminal used in a pharmacy in which a medicine is to be dispensed based on the prescription.

### Supplementary Note 5

The information providing apparatus according to any one of Supplementary Notes 1 to 4, in which
the predicting means predicts the time for the customer to wait based on a type of a medicine used for medicine dispensing based on the prescription.

### Supplementary Note 6

The information providing apparatus according to any one of Supplementary Notes 1 to 4, in which
the predicting means predicts the time for the customer to wait based on the information indicating the working status of medicine dispensing based on the prescription.

### Supplementary Note 7

The information providing apparatus according to any one of Supplementary Notes 1 to 6, in which
the outputting means determines content of the information to be provided to the customer in accordance with a length of the predicted time to wait.

### Supplementary Note 8

The information providing apparatus according to any one of Supplementary Notes 1 to 7, in which
an authenticating means for authenticating the customer by using authentication information issued based on the information regarding the prescription is further included.

### Supplementary Note 9

The information providing apparatus according to Supplementary Note 8, in which
the identifying means does not identify the customer when authentication of the customer is failed.

### Supplementary Note 10

The information providing apparatus according to any one of Supplementary Notes 1 to 9, in which
an estimating means for estimating a physical condition of the customer based on at least one of biological information of the customer and the information regarding the prescription is further included,
in which, when the customer is estimated to be in a bad physical condition, the outputting means is not allowed to output the information to be provided to the customer even when the predicted time for the customer to wait exceeds the threshold value.

### Supplementary Note 11

The information providing apparatus according to Supplementary Note 10, in which
the outputting means determines content of the information to be provided to the customer based on the estimated physical condition of the customer.

### Supplementary Note 12

An information providing method causes a computer to:
identify a customer;
acquire information regarding a prescription issued to the identified customer and information indicating a work status of medicine dispensing based on the prescription;
predict a time for the customer to wait based on at least one of the information regarding the prescription and the information indicating the work status of medicine dispensing based on the prescription; and
output information to be provided to the customer when the predicted time for the customer to wait exceeds a threshold value.

### Supplementary Note 13

A program causes a computer to execute:
processing for identifying a customer;
processing for acquiring information regarding a prescription issued to the identified customer and information indicating a work status of medicine dispensing based on the prescription;
processing for predicting a time for the customer to wait based on at least one of the information regarding the prescription and the information indicating the work status of medicine dispensing based on the prescription; and
processing for outputting information to be provided to the customer when the predicted time for the customer to wait exceeds a threshold value.

Some or all of the configurations according to Supplementary Notes 2 to 11 that are dependent on Supplementary Note 1 (the information providing apparatus) described above may also be dependent on Supplementary Note 12 (the information providing method) and Supplementary Note 13 (the program) due to a dependency relationship similar or identical to the dependency relationships of Supplementary Notes 2 to 11. Some or all of the configurations described as Supplementary Notes may be similarly or identically dependent on various hardware, various software, various recording means for recording the software, or various systems, without departing from the embodiments described above.

1. An information providing apparatus comprising:
a memory storing instructions; and
one or more processors configured to execute the instructions to:
identify a customer;
acquire information regarding a prescription issued to the identified customer and information indicating a work status of medicine dispensing based on the prescription;
predict a time for the customer to wait based on at least one of the information regarding the prescription and the information indicating the work status of medicine dispensing based on the prescription; and
output information to be provided to the customer when the predicted time for the customer to wait exceeds a threshold value.

2. The information providing apparatus according to claim 1, wherein
the one or more processors are further configured to execute the instructions to:
identify the customer by using biological information of the customer.

3. The information providing apparatus according to claim 1, wherein
the one or more processors are further configured to execute the instructions to:
acquire the information regarding the prescription from an electronic prescription corresponding to the prescription or image data acquired by photographing the prescription.

4. The information providing apparatus according to claim 1, wherein
the one or more processors are further configured to execute the instructions to:
predict the time for the customer to wait based on a type of a medicine used for medicine dispensing based on the prescription.

5. The information providing apparatus according to claim 1, wherein
the one or more processors are further configured to execute the instructions to:
predict the time for the customer to wait based on the information indicating the work status of medicine dispensing based on the prescription.

6. The information providing apparatus according to claim 1, wherein
the one or more processors are further configured to execute the instructions to:
determine content of the information to be provided to the customer in accordance with a length of the predicted time to wait.

7. The information providing apparatus according to claim 1, wherein
the one or more processors are further configured to execute the instructions to:
authenticate the customer by using authentication information issued based on the information regarding the prescription.

8. The information providing apparatus according to claim 1, wherein
the one or more processors are further configured to execute the instructions to:
estimate a physical condition of the customer based on at least one of biological information of the customer and the information regarding the prescription; and
allow, in a case where the customer is estimated to be in a bad physical condition, not to output the information to be provided to the customer even when the predicted time for the customer to wait exceeds the threshold value.

9. An information providing method by a computer, the information providing method comprising:
identifying a customer;
acquiring information regarding a prescription issued to the identified customer and information indicating a work status of medicine dispensing based on the prescription;
predicting a time for the customer to wait based on at least one of the information regarding the prescription and the information indicating the work status of medicine dispensing based on the prescription; and
outputting information to be provided to the customer in a case where the predicted time for the customer to wait exceeds a threshold value.

10. A non-transitory computer-readable recording medium that records a program for causing a computer to execute:
identifying a customer;
acquiring information regarding a prescription issued to the identified customer and information indicating a work status of medicine dispensing based on the prescription;
predicting a time for the customer to wait based on at least one of the information regarding the prescription and the information indicating the work status of medicine dispensing based on the prescription; and
outputting information to be provided to the customer in a case where the predicted time for the customer to wait exceeds a threshold value.
